SEQUENCE LISTING (98 sequences)

SEQ ID NO: 1 - DNA, 18 bp, Artificial Sequence
  HLA-A primer 1
ggagtattgggaccggaa

SEQ ID NO: 2 - DNA, 18 bp, Artificial Sequence
  HLA-A primer 2
gtggcccctggtacccgt

SEQ ID NO: 3 - DNA, 19 bp, Artificial Sequence
  HLA-A primer 3
acggaatgtgaaggcccag

SEQ ID NO: 4 - DNA, 19 bp, Artificial Sequence
  HLA-A primer 4
cctccaggtaggctctcaa

SEQ ID NO: 5 - DNA, 17 bp, Artificial Sequence
  HLA-A primer 5
agcgacgccgcgagcca

SEQ ID NO: 6 - DNA, 18 bp, Artificial Sequence
  HLA-A primer 6
cgtcgtaggcgtcctgcc

SEQ ID NO: 7 - DNA, 19 bp, Artificial Sequence
  HLA-A primer 7
ggccggagtattgggacga

SEQ ID NO: 8 - DNA, 18 bp, Artificial Sequence
  HLA-A primer 8
ctggtaccggcggaggag

SEQ ID NO: 9 - DNA, 19 bp, Artificial Sequenc
  HLA-A primer 9
gatagagcaggagaggcct

SEQ ID NO: 10 - DNA, 19 bp, Artificial Sequence
  HLA-A primer 10
cggaatgtgaaggcccact

SEQ ID NO: 11 - DNA, 17 bp, Artificial Sequence
  HLA-A primer 11
cccggcccggcagtgga

SEQ ID NO: 12 - DNA, 19 bp, Artificial Sequence
  HLA-A primer 12
gtggatagagcaggagggt

SEQ ID NO: 13 - DNA, 21 bp, Artificial Sequence
  HLA-A primer 13
atgtaatccttgccgtcgtaa

SEQ ID NO: 14 - DNA, 20 bp, Artificial Sequence
  HLA-A primer 14
ggaccaggagacacggaata

SEQ ID NO: 15 - DNA, 18 bp, Artificial Sequence
  HLA-A primer 15
cactccacgcacgtgcca

SEQ ID NO: 16 - DNA, 20 bp, Artificial Sequence
  HLA-A primer 16
tcacagactgaccgagagag

SEQ ID NO: 17 - DNA, 19 bp, Artificial Sequence
  HLA-A primer 17
agcgcaggtcctcgttcaa

SEQ ID NO: 18 - DNA, 18 bp, Artificial Sequence
  HLA-A primer 18
ccgtcgtaggcgtgctgt

SEQ ID NO: 19 - DNA, 19 bp, Artificial Sequence
  HLA-A primer 19
cacgcagttcgtgcggttt

SEQ ID NO: 20 - DNA, 18 bp, Artificial Sequence
  HLA-A primer 20
ctctctgctgctccgccg

SEQ ID NO: 21 - DNA, 18 bp, Artificial Sequence
  HLA-A primer 21
caagagcgcaggtcctcg

SEQ ID NO: 22 - DNA, 18 bp, Artificial Sequence
  HLA-A primer 22
caagagcgcaggtcctcg

SEQ ID NO: 23 - DNA, 19 bp, Artificial Sequence
  HLA-A primer 23
cggaatgtgaaggcccagt

SEQ ID NO: 24 - DNA, 19 bp, Artificial Sequence
  HLA-A primer 24
cctccaggtaggctctctg

SEQ ID NO: 25 - DNA, 18 bp, Artificial Sequence
  HLA-A primer 25
ccgagtggacctggggac

SEQ ID NO: 26 - DNA, 18 bp, Artificial Sequence
  HLA-A primer 26
gagccactccacgcacgt

SEQ ID NO: 27 - DNA, 19 bp, Artificial Sequence
  HLA-A primer 27
actcacagactgaccgagc

SEQ ID NO: 28 - DNA, 18 bp, Artificial Sequence
  HLA-A primer 28
aggatggagccgcgggca

SEQ ID NO: 29 - DNA, 18 bp, Artificial Sequence
  HLA-A primer 29
aggtatctgcggagcccg

SEQ ID NO: 30 - DNA, 18 bp, Artificial Sequence
  HLA-A primer 30
tcctcgtccccaggctct

SEQ ID NO: 31 - DNA, 18 bp, Artificial Sequence
  HLA-A primer 31
gagccactccacgcaccg

SEQ ID NO: 32 - DNA, 19 bp, Artificial Sequence
  HLA-A primer 32
acctgcggatcgcgctccg

SEQ ID NO: 33 - DNA, 18 bp, Artificial Sequence
  HLA-A primer 33
gggtaccagcaggacgct

SEQ ID NO: 34 - DNA, 18 bp, Artificial Sequence
  HLA-A primer 34
ggaccactccacgcactc

SEQ ID NO: 35 - DNA, 21 bp, Artificial Sequence
  Homo sapiens adenomatous polyposis coli(APC) primer 35
atgatgttgacctttccaggg

SEQ ID NO: 36 - DNA, 24 bp, Artificial Sequence
  Homo sapiens adenomatous polyposis coli (APC) primer 36
attctgtaacttttcatcagttgc

SEQ ID NO: 37 - DNA, 31 bp, Artificial Sequence
  HLA-A probe 1
cccggtttcattttcagtttaggccaaaaat

SEQ ID NO: 38 - DNA, 30 bp, Artificial Sequence
  HLA-A probe 2
gttctcacaccvtccagakratgtdtggct

SEQ ID NO: 39 - DNA, 19 bp, Artificial Sequence
  HLA-B primer 39
ggcgccgtggatagagcaa

SEQ ID NO: 40 - DNA, 20 bp, Artificial Sequence
  HLA-B primer 40
ccactccatgaggtatttcc

SEQ ID NO: 41 - DNA, 18 bp, Artificial Sequence
  HLA-B primer 41
cgccacgagtccgaggaa

SEQ ID NO: 42 - DNA, 18 bp, Artificial Sequence
  HLA-B primer 42
cgcgagtccgagagagga

SEQ ID NO: 43 - DNA, 17 bp, Artificial Sequence
  HLA-B primer 43
gccgcgagtccgaggac

SEQ ID NO: 44 - DNA, 18 bp, Artificial Sequence
  HLA-B primer 44
cgcgagtccgaggatggc

SEQ ID NO: 45 - DNA, 20 bp, Artificial Sequence
  HLA-B primer 45
gaccggaacacacagatctg

SEQ ID NO: 46 - DNA, 19 bp, Artificial Sequence
  HLA-B primer 46
accgggagacacagatctg

SEQ ID NO: 47 - DNA, 19 bp, Artificial Sequence
  HLA-B primer 47
accgggagacacagatctc

SEQ ID NO: 48 - DNA, 19 bp, Artificial Sequence
  HLA-B primer 48
ggagtattgggaccggaac

SEQ ID NO: 49 - DNA, 19 bp, Artificial Sequence
  HLA-B primer 49
gaacatgaaggcctccgcg

SEQ ID NO: 50 - DNA, 20 bp, Artificial Sequence
  HLA-B primer 50
gaccggaacacacagatctt

SEQ ID NO: 51 - DNA, 19 bp, Artificial Sequence
  HLA-B primer 51
gacgacacccagttcgtga

SEQ ID NO: 52 - DNA, 18 bp, Artificial Sequence
  HLA-B primer 52
taccgagagaacctgcgc

SEQ ID NO: 53 - DNA, 18 bp, Artificial Sequence
  HLA-B primer 53
gagcaggaggggccggaa

SEQ ID NO: 54 - DNA, 18 bp, Artificial Sequence
  HLA-B primer 54
ggccggagtattgggacg

SEQ ID NO: 55 - DNA, 19 bp, Artificial Sequence
  HLA-B primer 55
gacgacacgcagttcgtga

SEQ ID NO: 56 - DNA, 19 bp, Artificial Sequence
  HLA-B primer 56
gacgacacgctgttcgtga

SEQ ID NO: 57 - DNA, 19 bp, Artificial Sequence
  HLA-B primer 57
gacggcacccagttcgtga

SEQ ID NO: 58 - DNA, 16 bp, Artificial Sequence
  HLA-B primer 58
gcgggcgccgtgggtg

SEQ ID NO: 59 - DNA, 20 bp, Artificial Sequence
  HLA-B primer 59
gaccggaacacacagatcta

SEQ ID NO: 60 - DNA, 19 bp, Artificial Sequence
  HLA-B primer 60
cagatctacaaggcccagg

SEQ ID NO: 61 - DNA, 18 bp, Artificial Sequence
  HLA-B priemr 61
ccgagagagcctgcggaa

SEQ ID NO: 62 - DNA, 19 bp, Artificial Sequence
  HLA-B primer 62
accgagagaacctgcggat

SEQ ID NO: 63 - DNA, 18 bp, Artificial Sequence
  HLA-B primer 63
gcgctccgctactacaac

SEQ ID NO: 64 - DNA, 19 bp, Artificial Sequence
  HLA-B primer 64
acgccgcgagtccgacagg

SEQ ID NO: 65 - DNA, 18 bp, Artificial Sequence
  HLA-B primer 65
ctcctgctgctctcggga

SEQ ID NO: 66 - DNA, 18 bp, Artificial Sequence
  HLA-B primer 66
cgccgcgagtccgagaga

SEQ ID NO: 67 - DNA, 20 bp, Artificial Sequence
  HLA-B primer 67
gagacacagaagtacaagcg

SEQ ID NO: 68 - DNA, 19 bp, Artificial Sequence
  HLA-B primer 68
accggaacacacagatctc

SEQ ID NO: 69 - DNA, 19 bp, Artificial Sequence
  HLA-B primer 69
cctccaggtaggctctgtc

SEQ ID NO: 70 - DNA, 17 bp, Artificial Sequence
  HLA-B primer 70
ggaggaggcgcccgtcg

SEQ ID NO: 71 - DNA, 18 bp, Artificial Sequence
  HLA-B primer 71
ccttgccgtcgtaggcgg

SEQ ID NO: 72 - DNA, 19 bp, Artificial Sequence
  HLA-B primer 72
atccttgccgtcgtaggct

SEQ ID NO: 73 - DNA, 20 bp, Artificial Sequence
  HLA-B primer 73
cgttcagggcgatgtaatct

SEQ ID NO: 74 - DNA, 17 bp, Artificial Sequence
  HLA-B primer 74
gccgcggtccaggagct

SEQ ID NO: 75 - DNA, 17 bp, Artificial Sequence
  HLA-B primer 75
gagccgccgtgtccgcg

SEQ ID NO: 76 - DNA, 18 bp, Artificial Sequence
  HLA-B primer 76
cgtgccctccaggtaggt

SEQ ID NO: 77 - DNA, 18 bp, Artificial Sequence
  HLA-B primer 77
gagccactccacgcactc

SEQ ID NO: 78 - DNA, 18 bp, Artificial Sequence
  HLA-B primer 78
gagccactccacgcacag

SEQ ID NO: 79 - DNA, 18 bp, Artificial Sequence
  HLA-B primer 79
ccaggtatctgcggagcg

SEQ ID NO: 80 - DNA, 18 bp, Artificial Sequence
  HLA-B primer 80
gagccactccacgcacgt

SEQ ID NO: 81 - DNA, 17 bp, Artificial Sequence
  HLA-B primer 81
ccgcgcgctccagcgtg

SEQ ID NO: 82 - DNA, 18 bp, Artificial Sequence
  HLA-B primer 82
taccagcgcgctccagct

SEQ ID NO: 83 - DNA, 18 bp, Artificial Sequence
  HLA-B primer 83
gggccgcctcccacttga

SEQ ID NO: 84 - DNA, 19 bp, Artificial Sequence
  HLA-B primer 84
cgtcgcagccatacatcca

SEQ ID NO: 85 - DNA, 20 bp, Artificial Sequence
  HLA-B primer 85
gccatacatcctctggatga

SEQ ID NO: 86 - DNA, 19 bp, Artificial Sequence
  HLA-B primer 86
cgtcgcagccatacatcac

SEQ ID NO: 87 - DNA, 19 bp, Artificial Sequence
  HLA-B primer 87
cgctctggttgtagtagcc

SEQ ID NO: 88 - DNA, 19 bp, Artificial Sequence
  HLA-B primer 88
cgctctggttgtagtagcg

SEQ ID NO: 89 - DNA, 18 bp, Artificial Sequence
  HLA-B primer 89
cgcgcgctgcagcgtctc

SEQ ID NO: 90 - DNA, 18 bp, Artificial Sequence
  HLA-B primer 90
gtcgtaggcgtactggtt

SEQ ID NO: 91 - DNA, 18 bp, Artificial Sequence
  HLA-B primer 91
gtcgtaggcgtactggtc

SEQ ID NO: 92 - DNA, 19 bp, Artificial Sequence
  HLA-B primer 92
caaacatcctctggagggt

SEQ ID NO: 93 - DNA, 20 bp, Artificial Sequence
  HLA-B primer 93
agtctgtgtgttggtcttgt

SEQ ID NO: 94 - DNA, 17 bp, Artificial Sequence
  HLA-B primer 94
gccgcggtccaggagct

SEQ ID NO: 95 - DNA, 20 bp, Artificial Sequence
  HLA-B primer 95
gccatacatcctctggatga

SEQ ID NO: 96 - DNA, 29 bp, Artificial Sequence
  HLA-B probe 3
accctcgaccggcgagagccccaggcgcg

SEQ ID NO: 97 - DNA, 28 bp, Artificial Sequence
  HLA-B probe 4
agagcargaggggccggartattgggac

SEQ ID NO: 98 - DNA, 29 bp, Artificial Sequence
  Homo sapiens adenomatous polyposis coli (APC) probe 2
acagaactaacctccaaccaacaatcagc

(19) United States
(12) Patent Application Publication
(10) Pub. No.: US 2014/0248613 A1
(43) Pub. Date: Sep. 4, 2014

(54) AUTOMATIC DETECTION KIT FOR DETECTING HLA ALLELES USING REAL-TIME POLYMERASE CHAIN REACTION

(71) Applicant: Catholic University Industry Academic Cooperation Foundation, Seoul (KR)

(72) Inventors: Tai Gyu KIM, Seoul (KR); Hee Baeg Choi, Seoul (KR)

(73) Assignee: Catholic University Industry Academic Cooperation Foundation, Seoul (KR)

(21) Appl. No.: 14/184,257

(22) Filed: Feb. 19, 2014

Related U.S. Application Data
(63) Continuation of application No. 13/376,075, filed on Dec. 2, 2011, now abandoned.

(30) Foreign Application Priority Data
Jun. 5, 2009 (KR) 10-2009-0050003
May 12, 2010 (KR) 10-2010-0044457

(51) Int. Cl. C12Q 1/68

(52) U.S. Cl. CPC C12Q 1/6881
USPC 435/6.11

(57) ABSTRACT

Provided is an automatic detection kit for automatically detecting HLA alleles using a real-time polymerase chain reaction (PCR). The real-time PCR is performed on DNA isolated from a sample using a primer which is able to specifically bind to HLA alleles and a fluorescent probe which is able to detect amplification of the HLA alleles in real time, and the HLA allele typing is performed by analyzing a fluorescence value obtained from the real-time PCR using an HLA automatic typing.

### CROSS-REFERENCE TO RELATED APPLICATION

This application claims priority to and the benefit of Korean Patent Application No. 2009-0050003, filed Jun. 5, 2009, and 2010-0044457, filed May 12, 2010, the disclosure of which is incorporated herein by reference in its entirety. This application is a Continuation of U.S. patent application Ser. No. 13/376,075, filed Dec. 2, 2011.

### BACKGROUND

1. Field of the Invention

The present invention relates to an automatic detection kit for detecting HLA alleles using a real-time polymerase chain reaction (PCR). More particularly, the present invention relates to an automatic detection kit for detecting HLA alleles using a real-time PCR, wherein the real-time PCR is performed on DNA isolated from a sample using a primer which is able to specifically bind to HLA alleles and a fluorescent probe which is able to detect amplification of the HLA alleles in real time, and the HLA allele typing is performed by analyzing a fluorescence value obtained from the real-time PCR using an HLA automatic typing program.

2. Discussion of Related Art

A human leukocyte antigen (HLA) is a human major histocompatibility complex (hMHC) which is a cell surface antigen generally associated with graft rejection. As transplantation is developed as a therapeutic method for treating incurable diseases with the development of modern medical science, the importance of the transplantation and demand for exact examination have increased. HLA genes are classified into class I, II and III loci. HLA-A, -B and -C genes are present in the HLA class I locus, and HLA-DR, DQ and DP genes are present in the HLA class II locus. HLA-A, B and C molecules are membrane glycoproteins, each of which is composed of three domains and expressed by binding to 13-2 microglobulin having a molecular weight of 12 kDa under the control of the 15^{th} chromosome. HLA-DR, DQ and DP molecules are present in a shape in which a and 13 chains interact to form a heterodimer, and a gene controlling these molecules is positioned 500 kb from a central end of MHC. Research on the diversity of HLA alleles has progressed rapidly as base sequences of HLA alleles have been found and the HLA allele DNA typing can be performed using a polymerase chain reaction (PCR). To date, a large number of the HLA alleles have been found, that is, 767 HLA-A, 1178 HLA-B and 440 HLA-C genes have been found in class I, and 3 HLA-DRA1, 618 HLA-DRB1, 34 HLA-DQA1, 96 HLA-DQB1, 27 HLA-DPA1 and 133 HLA-DPB1 genes have been found in class II. In the past several years, most studies on HLA genes have been converted into DNA testing methods. Recently, the DNA testing methods on the HLA genes have been widely used and developed. This is because there is an increased necessity to convert them into a high-accuracy DNA testing method having high accuracy due to the inaccuracy in a serological test, the DNA testing method is relatively easy in a technical aspect, and a simple and accurate product has been on sale as a kit. A currently commercialized test kit for testing HLA gene types has been developed using a nylon membrane, a PCR-sequence-specific oligonucleotide (PCR-SSO), a reverse blot PCR-SSO, a PCR-sequence-specific primer (SSP), a PCR-oligocapture sandwich assay, a luminex bead array method and a PCR-sequence-based typing (SBT) method. However, the conventional methods have problems in that a user basically has to perform a PCR and then perform other reactions such as hybridization and electrophoresis to perform an HLA test, which is inconvenient and time-consuming. Therefore, there is a continuous demand for methods, such as a real-time PCR, by which the HLA test can be performed using only a PCR.

When an HLA test is performed using only the real-time PCR, amplification of HLA genes is determined using a primer labeled with a fluorescent material. In this case, when single-base mutations are present in one gene, several corresponding fluorescent primers are required to detect the single-base mutations. Therefore, it is difficult to perform such research since a gene containing numerous single-base mutations requires various kinds of expensive fluorescent probes.

Therefore, there is a demand for an HLA testing/typing method using a minimum quantity of an expensive fluorescent probe and a real-time PCR.

### SUMMARY OF THE INVENTION

The present invention is directed to providing a selection kit for detecting a locus having genetic polymorphisms which is able to specifically amplify the locus having the genetic polymorphisms using a conventional primer free of a fluorescent material and analyze the HLA allele typing in real time using a fluorescent probe which is able to detect amplification of the locus only, and a method for typing HLA alleles using the same.

In one aspect, a detection set for detecting HLA alleles using a real-time polymerase chain reaction (PCR), which includes a primer which is able to specifically amplify the HLA allele, includes at least one fluorescent probe selected from the group consisting of sequences set forth in SEQ ID NOS: 37, 38, 96 and 97, which are able to detect amplification of the HLA alleles.

In another aspect, a selection kit for typing HLA alleles includes a detection set according to the present invention.

In still another aspect, a method for typing HLA alleles includes performing a real-time PCR on DNA isolated from a sample using a detection set for detecting HLA alleles according to the present invention, and typing the HLA alleles by checking the real-time PCR results.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIGS. 1 and 2 show an analysis table and analysis results for classifying HLA-A allele types, and

FIGS. 3 to 6 show an analysis table and analysis results for classifying HLA-B allele types.

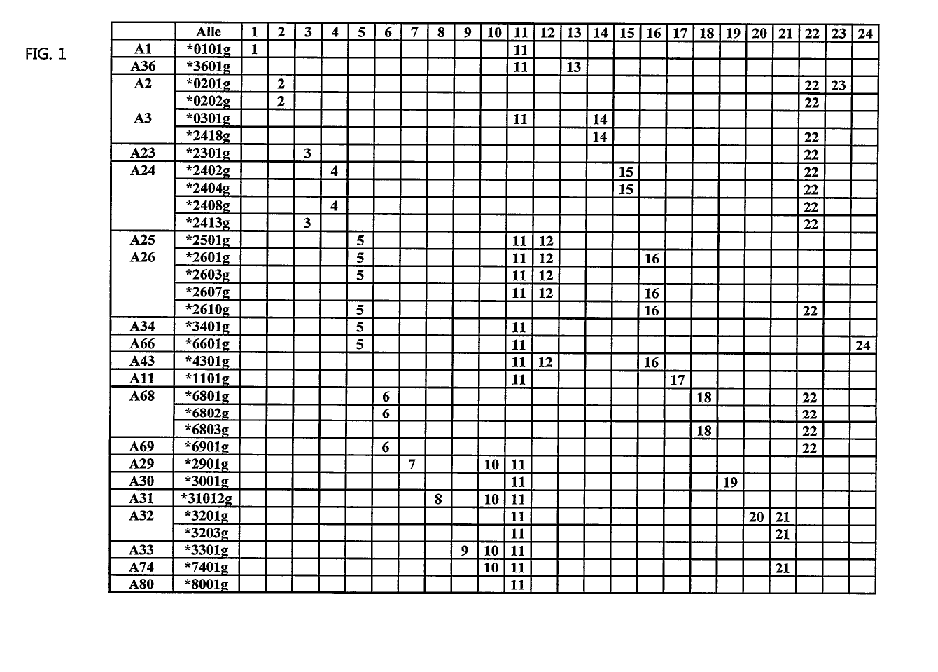

### DETAILED DESCRIPTION OF EXEMPLARY EMBODIMENTS

Hereinafter, the present invention will be described in detail with respect to Examples according to the present invention and Comparative Examples not according to the present invention, but the scope of the present invention is not limited thereto.

The present invention relates to a detection set for detecting HLA alleles using a real-time PCR, which includes a primer which is able to specifically amplify the HLA alleles. Here, the detection set includes at least one fluorescent probe selected from the group consisting of sequences set forth in SEQ ID NO: 37, 38, 96 and 97 which is able to detect amplification of the HLA alleles.

The detection set for detecting HLA alleles using the real-time PCR according to the present invention may detect the HLA alleles through the real-time PCR using both a primer which is able to specifically bind to the HLA alleles and amplify genes of the HLA alleles using the real-time PCR and a fluorescence-labeled probe which is hybridizable with a PCR product.

The HLA alleles which can be detected using the detection set according to the present invention may be HLA-A or HLA-B alleles.

A real-time PCR method disclosed herein refers to a method of quantifying target DNA by monitoring generation of a PCR product in real time using a device integrally formed with a thermal cycler and a spectrofluorophotometer. The real-time PCR method does not require electrophoresis to determine the presence of the PCR product, and has excellent rapidity and quantitative property since it can quantify the PCR product more accurately by comparing a capacity of the PCR product in a region in which the amplification takes place in an exponential function.

The primer may be used to specifically amplify the HLA alleles, depending on the kinds of the HLA alleles. According to one exemplary embodiment, the primer may be constructed to have a length of 17 to 24 nucleotides (nt) using a locus showing the diversity of the HLA alleles as the starting point.

The primer may amplify the HLA-A allele, such as, for example, HLA-A*01, A*02, A*03, A*11, A*23, A*24, A*25, A*26, A*29, A*30, A*31, A*32, A*33, A*34, A*43, A*66, A*68, A*69, A*74, or A*80; or HLA-B allele, such as, for example, HLA-B*27, B60(*4001group), B61(*4002group), B40(*4008group), B*47, B841, B*13, B*44, B*45, B*49, B*40, B*54, B*59, B*55, B*56, B*38, B*39, B*67, B*14, B*07, B*08, B*18, B*37, B*35, B*53, B*51, B*52, B*57, B*58, B62(*1501 group), B75(*1502group), B72(*1503group), B76(*1512group), B63(*1516group), B77(*1513group), B*46, B*48, B71(*1509group), B*78, B*81, 5 B*82, or B*83 using a conventional PCR. More preferably, the primer may be used to amplify the HLA alleles using a single or multiplex real-time PCR.

According to one exemplary embodiment, a size of a PCR product which may be amplified using the primer may be in a range of 171 to 813 bp in consideration of the conditions in which a non-specific reaction does not take place. In this case, a Tm value of all the primers may be in a range of 58 to 60° C.

The kinds of HLA-A alleles which are able to specifically amplify the respective primers are as follows.
1)
   Primer pair: SEQ ID NOS: 14 and 29
   Detected HLA allele: A*01
2)
   Primer pair: SEQ ID NOS: 30 and 2
   Detected HLA allele: A*02
3)
   Primer pair: SEQ ID NOS: 7 and 4
   Detected HLA alleles: A*23 and A*24
4)
   Primer pair: SEQ ID NOS: 32 and 24
   Detected HLA allele: A*24
5)
   Primer pair: SEQ ID NOS: 1 and 13
   Detected HLA alleles: A*25, A*26, A*34 and A*66
6)
   Primer pair: SEQ ID NOS: 3 and 22
   Detected HLA alleles: A*68 and A*69
7)
   Primer pair: SEQ ID NOS: 28 and 17
   Detected HLA allele: A*29
8)
   Primer pair: SEQ ID NOS: 9 and 17
   Detected HLA allele: A*31
9)
   Primer pair: SEQ ID NOS: 1 and 17
   Detected HLA allele: A*33
10)
   Primer pair: SEQ ID NOS: 25 and 17
   Detected HLA alleles: A*29, A*31, A*33 and A*74
11)
   Primer pair: SEQ ID NOS: 10, 23 and 21
   Detected HLA alleles: A*01, A*03, A*25, A*26, A*34, A*66, A*43, A*11, A*29, A*30, A*31, A*32, A*33, A*74 and A*80
12)
   Primer pair: SEQ ID NOS: 33 and 31
   Detected HLA alleles: A*25, A*26 and A*43
13)
   Primer pair: SEQ ID NOS: 14 and 26
   Detected HLA allele: A*36
14)
   Primer pair: SEQ ID NOS: 5 and 15
   Detected HLA alleles: A*03 and A*24
15)
   Primer pair: SEQ ID NOS: 7 and 24
   Detected HLA allele: A*24
16)
   Primer pair: SEQ ID NOS: 27 and 13
   Detected HLA alleles: A*26 and A*43
17)
   Primer pair: SEQ ID NOS: 3 and 20
   Detected HLA allele: A*11
18)
   Primer pair: SEQ ID NOS: 1 and 6
   Detected HLA allele: A*68
19)
   Primer pair: SEQ ID NOS: 11 and 18
   Detected HLA allele: A*30
20)
   Primer pair: SEQ ID NOS: 16 and 17
   Detected HLA allele: A*32
21)
   Primer pair: SEQ ID NOS: 19 and 8
   Detected HLA alleles: A*32 and A*74
22)
   Primer pair: SEQ ID NOS: 10, 23 and 22
   Detected HLA alleles: A*02, A*24, A*26, A*68 and A*69
23)
   Primer pair: SEQ ID NOS: 12 and 4
   Detected HLA allele: A*02
24)
   Primer pair: SEQ ID NOS: 33 and 34
   Detected HLA allele: A*66

Also, the kinds of HLA-B alleles which are able to specifically amplify the respective primers are as follows.
1)
   Primer pair: SEQ ID NOS: 46 and 77
   Detected HLA allele: B*27(*2701g)
2)
   Primer pair: SEQ ID NOS: 41 and 75
   Detected HLA allele: B60(*4001g), B61(*4002g), B40(*4008g) and B47(*4701 g)
3)
   Primer pair: SEQ ID NOS: 49 and 78
   Detected HLA alleles: B57(*5701g), B58(*5801g) and B63(*1516g)
      [114]
4)
   Primer pair: SEQ ID NOS: 42 and 82
   Detected HLA alleles: B7(*0702g), B48(*4801g) and B81(*8101g)
      [117]
5)
   Primer pair: SEQ ID NOS: 52 and 83
   Detected HLA allele: B13(*1301g))
6)
   Primer pair: SEQ ID NOS: 41 and 89
   Detected HLA alleles: B61(*4002g), B40(*4008g), B47(*4701g), B44(*4402g), B45(*4501g), B49(*4901g) and B50(*5001g)
7)
   Primer pair: SEQ ID NOS: 47 and 79
   Detected HLA alleles: B44(*4402g) and B45(*4501g)
8)
   Primer pair: SEQ ID NOS: 40 and 72
   Detected HLA alleles: B45(*4501g), B49(*4901g) and B50(*5001g)
9)
   Primer pair: SEQ ID NOS: 48 and 72
   Detected HLA alleles: B54(*5401g), B59(*5901g), B55(*5001g), B56(5601g) and B82(*8201g)
10)
   Primer pair: SEQ ID NOS: 53 and 76
   Detected HLA alleles: B38(*3801g), B39(*3901g) and B67(*6701g)
      [135]
11)
   Primer pair: SEQ ID NOS: 53 and 84
   Detected HLA allele: B14(*1401g)
12)
   Primer pair: SEQ ID NOS: 48 and 82
   Detected HLA alleles: B40(*4025g), B7(*0702g) and B81(*8101g)
13)
   Primer pair: SEQ ID NOS: 50 and 81
   Detected HLA allele: B8(*0801g)
14)
   Primer pair: SEQ ID NOS: 39 and 74
   Detected HLA allele: B18(*1801g)
15)
   Primer pair: SEQ ID NOS: 43 and 69
   Detected HLA alleles: B37(*3701g) and B51(*5108g)
16)
   Primer pair: SEQ ID NOS: 50 and 70
   Detected HLA alleles: B35(*3501g) and B53(*5301g)
17)
   Primer pair: SEQ ID NOS: 43 and 73
   Detected HLA alleles: B51(*5101g) and B52(*5201g)
18)
   Primer pair: SEQ ID NOS: 54 and 86
   Detected HLA allele: B57(*5701g)
19)
   Primer pair: SEQ ID NOS: 49 and 85
   Detected HLA alleles: B57(*5705g) and B58(*5801g)
20)
   Primer pair: SEQ ID NOS: 47 and 71
   Detected HLA alleles: B13(*1304g), B61(*4003g), B62(*1501g), B72(*1503g) and B76(*1512g)
21)
   Primer pair: SEQ ID NOS: 44 and 78
   Detected HLA alleles: B57(*5701g), B62(*1501g), B75(*1502g), B63(*1516g), B77(*1513g) and B46(*4601g)
22)
   Primer pair: SEQ ID NOS: 51 and 81
   Detected HLA allele: B42(*4201g)
23)
   Primer pair: SEQ ID NOS: 47 and 82
   Detected HLA alleles: B60(*4001g) and B48(*4801g)
24)
   Primer pair: SEQ ID NOS: 45 and 71
   Detected HLA alleles: B39(*3907g) and B75(*1521g)
25)
   Primer pair: SEQ ID NOS: 58 and 74
   Detected HLA allele: B54(*5401g)
26)
   Primer pair: SEQ ID NOS: 55, 56, 51, 57 and 88
   Detected HLA alleles: B27(*2701g), B47(*4701g), B13(*1301g), B44(*4402g), B49(*4901g), B59(*5901 g), B38(*3801g), B8(*0802g), B18(*1809g), B37(*3701g), B53(*5301g), B51(*5101g), B52(*5201g), B57(*5701g), B58(*5801g), B63(*1516g), B77(*1513g) and B62(*1524g)
27)
   Primer pair: SEQ ID NOS: 55, 56, 51, 57 and 87
   Detected HLA alleles: B27(*2708g), B60(*4001g), B61(*4002g), B40(*4008g), B47(*4702g), B41(*4101g), B44(*4409g), B45(*4501g), B50(*5001g), B55(*5501g), B56(*5601g), B39(*3901g), B67(*6701g), B14(*1401g), B7(*0702g), B8(*0801g), B18(*1801g), B37(*3705g), B35(*3501g), B62(*1501g), B15(*1530g), B72(*1503g), B76(*1512g), B46(*4601g), B42(*4201g), B48(*4801g), B71(*1509g), B78(*7801g), B81(*8101g), B82(*8201g) and B83(*8301g)
28)
   Primer pair: SEQ ID NOS: 48 and 73
   Detected HLA alleles: B56(*5605g), B51(*5101g), B71(*1509g) and B78(*7801g)
29)
   Primer pair: SEQ ID NOS: 47 and 73
   Detected HLA alleles: B40(*4026g), B52(*5201g) and B62(*15012)
30)
   Primer pair: SEQ ID NOS: 60 and 78
   Detected HLA allele: B56(*5601g)
31)
   Primer pair: SEQ ID NOS: 59 and 76
   Detected HLA alleles: B39(*3910g) and B67(*67011g)
32)
   Primer pair: SEQ ID NOS: 62 and 72
   Detected HLA alleles: B49(*4901g) and B59(*5901g)
33)
   Primer pair: SEQ ID NOS: 61 and 76
   Detected HLA alleles: B39(*3901g), B67(*6701g) and B51(*5115g)
      [204]
34)
   Primer pair: SEQ ID NOS: 54 and 71
   Detected HLA alleles: B57(*5701g) and B58(*5801g)
35)
   Primer pair: SEQ ID NOS: 41 and 71
   Detected HLA alleles: B61(*4003g), B13(*1304g) and B72(*1546g)
36)
   Primer pair: SEQ ID NOS: 66 and 72
   Detected HLA alleles: B59(*5901g), B55(*5501g), B56(*5601g), B39(*3917g) and B82(*8201g)
37)
   Primer pair: SEQ ID NOS: 61 and 90
   Detected HLA alleles: B60(*4001g), B62(*4002g), B40(*4008g), B41(*4101g), B55(*5504g), B56(*5605g), B8(*0801g), B35(*3502g), B15(*1530g), B42(*4201g), B48(*4801g), B71(*1509g) and B78(*7801g)
38)
   Primer pair: SEQ ID NOS: 50 and 75
   Detected HLA alleles: B40(*4008g), B35(*3509g) and B48(*4806g)
39)
   Primer pair: SEQ ID NOS: 60 and 80
   Detected HLA alleles: B54(*5401g), B55(*5501g), B67(*6701g), B7(*0719g) and B42(*4201g)
40)
   Primer pair: SEQ ID NOS: 64 and 72
   Detected HLA alleles: B54(*5401g)
41)
   Primer pair: SEQ ID NOS: 45 and 76
   Detected HLA alleles: B38(*3801g) and B39(*3901g)
42)
   Primer pair: SEQ ID NOS: 63 and 76
   Detected HLA allele: B38(*3801g)
43)
   Primer pair: SEQ ID NOS: 60 and 91
   Detected HLA allele: B7(*0702g)
44)
   Primer pair: SEQ ID NOS: 44 and 82
   Detected HLA allele: B40(*4021g)
45)
   Primer pair: SEQ ID NOS: 41 and 92
   Detected HLA allele: B47(*4701g)
46)
   Primer pair: SEQ ID NOS: 65 and 93
   Detected HLA alleles: B71(*1509g) and B75(*1511g)
47)
   Primer pair: SEQ ID NOS: 67 and 94
   Detected HLA allele: B46(*4601g)
48)
   Primer pair: SEQ ID NOS: 68 and 95
   Detected HLA alleles: B35(*3520g), B75(*1502g) and B77(*1513g)

Also, a fluorescent probe may be constructed to have a length of 28 to 35 nt by selecting a polymorphism-free locus on an HLA gene to detect all products amplified using the primers. Particularly, the fluorescent probe may have a sequence set forth in SEQ ID NO: 37, 38, 96 or 97. More particularly, it is possible 10 to detect amplification of the HLA-A alleles using a fluorescent probe having a sequence set forth in SEQ ID NO: 37 or 38. Also, it is possible to detect amplification of the HLA-B alleles using a fluorescent probe having a sequence set forth in SEQ ID NO: 96 or 97. In this case, a Tm value of the fluorescent probes may be in a range of 60 to 65° C.

The fluorescent probes have the 5′ and 3′ termini labeled with a fluorescent marker. The respective fluorescent probes are labeled with different fluorescent markers according to the kind of detection sets for detecting HLA alleles. The fluorescent markers have different excitation and emission wavelengths according to the kind of markers, and thus the use of the fluorescent markers is also different. In consideration of these facts, a fluorescent marker used together with one PCR reaction product should be selected and used to determine whether the HLA alleles can be detected individually. Specific contents and selection of the fluorescent markers are obvious to those skilled in the art to which the present invention pertains.

According to one exemplary embodiment, probes included in the detection set for detecting HLA alleles according to the present invention are labeled with the fluorescent markers using a conventional method. A labeling method may include an interchelating method, a TaqMan™ probe method and a molecular beacon method. The TaqMan™ probe method used herein is performed by adding an oligonucleotide (TaqMan™ probe) whose 5′ terminus is labeled with a fluorescent marker (i.e., FAM) and whose 3′ terminus is labeled with a quencher material (i.e., TAMRA) to a PCR reaction solution. Here, the TaqMan™ probe specifically hybridizes with template DNA in an annealing process, but fluorescence generation is inhibited by a fluorescence quencher on the probe. In an extension process, only the TaqMan™ probe hybridized with the template is disintegrated by the 5′→3′ exonuclease activity of Taq DNA polymerase to release a fluorescent dye from the probe. Therefore, the inhibition by the fluorescent quencher is removed and the fluorescence is emitted. In this case, the probe has a 5′ terminus labeled with one fluorescent marker selected from the group consisting of FAM, VIC, TET, JOE, HEX, CY3, CY5, ROX, RED610, TEXAS RED, RED670 and NED, and a 3′ terminus labeled with one fluorescence quencher selected from the group consisting of 6-TAMRA, BHQ-1,2,3 and a molecular grove binding non-fluorescence quencher (MGBNFQ).

Also, the detection set according to the present invention may further include a primer for amplifying an internal positive-control gene to enhance test reliability and check the status of a template in all PCR tubes and the PCR conditions used in this experimental procedure. Preferably, 3-globin, human 3-actin, glyceraldehydes-3-phosphate dehydrogenase (GAPDH), or a Homo sapiens adenomatous polyposis coli (APC) gene may be used herein. More preferably, a primer pair having sequences set forth in SEQ ID NOS: 35 and 36, which are able to amplify the Homo sapiens APC gene may be used.

Therefore, the detection set according to the present invention may further include a fluorescent probe for detecting amplification of a positive-control gene. Preferably, the fluorescent probe having a sequence set forth in SEQ ID NO: 98 may be used herein.

According to one exemplary embodiment, the details of the detection set including primers and a fluorescent probe to amplify and detect the HLA-A alleles are as follows. Also, the same kinds of a positive control primer pair (SEQ ID NOS: 35 and 36) and a fluorescent probe (SEQ ID NO: 37, 38 or 98) are used in all the detection sets:
1)
   Primer pair: SEQ ID NOS: 14 and 29
   Size of PCR product: 574 by
2)
   Primer pair: SEQ ID NOS: 30 and 2
   Size of PCR product: 813 by
3)
   Primer pair: SEQ ID NOS: 7 and 4
   Size of PCR product: 555 by
4)
   Primer pair: SEQ ID NOS: 32 and 24
   Size of PCR product: 462 by
5)
   Primer pair: SEQ ID NOS: 1 and 13
   Size of PCR product: 438 by
6)
   Primer pair: SEQ ID NOS: 3 and 22
   Size of PCR product: 445 by
7)
   Primer pair: SEQ ID NOS: 28 and 17
   Size of PCR product: 515 by
8)
   Primer pair: SEQ ID NOS: 9 and 17
   Size of PCR product: 481 by
9)
   Primer pair: SEQ ID NOS: 1 and 17
   Size of PCR product: 461 by
10)
   Primer pair: SEQ ID NOS: 25 and 17
   Size of PCR product: 414 by
11)
   Primer pair: SEQ ID NOS: 10, 23 and 21
   Size of PCR product: 446 by
12)
   Primer pair: SEQ ID NOS: 33 and 31
   Size of PCR product: 171 by
13)
   Primer pair: SEQ ID NOS: 14 and 26
   Size of PCR product: 563 by
14)
   Primer pair: SEQ ID NOS: 5 and 15
   Size of PCR product: 626 by
15)
   Primer pair: SEQ ID NOS: 7 and 24
   Size of PCR product: 398 by
16)
   Primer pair: SEQ ID NOS: 27 and 13
   Size of PCR product: 400 by
17)
   Primer pair: SEQ ID NOS: 3 and 20
   Size of PCR product: 518 by
18)
   Primer pair: SEQ ID NOS: 1 and 6
   Size of PCR product: 426 by
19)
   Primer pair: SEQ ID NOS: 11 and 18
   Size of PCR product: 560 by
20)
   Primer pair: SEQ ID NOS: 16 and 17
   Size of PCR product: 421 by
21)
   Primer pair: SEQ ID NOS: 19 and 8
   Size of PCR product: 494 by
22)
   Primer pair: SEQ ID NOS: 10, 23 and 22
   Size of PCR product: 446 by
23)
   Primer pair: SEQ ID NOS: 12 and 4
   Size of PCR product: 574 by
24)
   Primer pair: SEQ ID NOS: 33 and 34
   Size of PCR product: 175 by

According to another exemplary embodiment, the details of the detection set including primers and a fluorescent probe to amplify and detect the HLA-B alleles are as follows. Also, the same kinds of a positive control primer pair (SEQ ID NOS: 35 and 36) and a fluorescent probe (SEQ ID NO: 96, 97 or 98) are used in all the detection sets:
1)
   Primer pair: SEQ ID NOS: 46 and 77
   Size of PCR product: 562 by
2)
   Primer pair: SEQ ID NOS: 41 and 75
   Size of PCR product: 544 by
3)
   Primer pair: SEQ ID NOS: 49 and 78
   Size of PCR product: 551 by
4)
   Primer pair: SEQ ID NOS: 42 and 82
   Size of PCR product: 548 by
5)
   Primer pair: SEQ ID NOS: 52 and 83
   Size of PCR product: 471 by
6)
   Primer pair: SEQ ID NOS: 41 and 89
   Size of PCR product: 669 by
7)
   Primer pair: SEQ ID NOS: 47 and 79
   Size of PCR product: 575 by
8)
   Primer pair: SEQ ID NOS: 40 and 72
   Size of PCR product: 600 by
9)
   Primer pair: SEQ ID NOS: 48 and 72
   Size of PCR product: 436 by
10)
   Primer pair: SEQ ID NOS: 53 and 76
   Size of PCR product: 572 by
11)
   Primer pair: SEQ ID NOS: 53 and 84
   Size of PCR product: 390 by
12)
   Primer pair: SEQ ID NOS: 48 and 82
   Size of PCR product: 619 by
13)
   Primer pair: SEQ ID NOS: 50 and 81
   Size of PCR product: 606 by
14)
   Primer pair: SEQ ID NOS: 39 and 74
   Size of PCR product: 503 by
15)
   Primer pair: SEQ ID NOS: 43 and 69
   Size of PCR product 606 by
16)
   Primer pair: SEQ ID NOS: 50 and 70
   Size of PCR product: 390 by
17)
   Primer pair: SEQ ID NOS: 43 and 73
   Size of PCR product: 504 by
18)
   Primer pair: SEQ ID NOS: 54 and 86
   Size of PCR product: 380 by
19)
   Primer pair: SEQ ID NOS: 49 and 85
   Size of PCR product: 345 by
20)
   Primer pair: SEQ ID NOS: 47 and 71
   Size of PCR product: 422 by
21)
   Primer pair: SEQ ID NOS: 44 and 78
   Size of PCR product: 623 by
22)
   Primer pair: SEQ ID NOS: 51 and 81
   Size of PCR product: 702 by
23)
   Primer pair: SEQ ID NOS: 47 and 82
   Size of PCR product: 608 by
24)
   Primer pair: SEQ ID NOS: 45 and 71
   Size of PCR product: 426 by
25)
   Primer pair: SEQ ID NOS: 58 and 74
   Size of PCR product: 508 by
26)
   Primer pair: SEQ ID NOS: 55, 56, 51, 57 and 88
   Size of PCR product: 180 by
27)
   Primer pair: SEQ ID NOS: 55, 56, 51, 57 and 87
   Size of PCR product: 180 by
28)
   Primer pair: SEQ ID NOS: 48 and 73
   Size of PCR product: 451 by
29)
   Primer pair: SEQ ID NOS: 47 and 73 25
   Size of PCR product: 440 by
30)
   Primer pair: SEQ ID NOS: 60 and 78
   Size of PCR product: 551 by
31)
   Primer pair: SEQ ID NOS: 59 and 76
   Size of PCR product: 548 by
32)
   Primer pair: SEQ ID NOS: 62 and 72
   Size of PCR product: 385 by
33)
   Primer pair: SEQ ID NOS: 61 and 76
   Size of PCR product: 567 by
34)
   Primer pair: SEQ ID NOS: 54 and 71
   Size of PCR product: 416 by
35)
   Primer pair: SEQ ID NOS: 41 and 71
   Size of PCR product: 487 by
36)
   Primer pair: SEQ ID NOS: 66 and 72
   Size of PCR product: 489 by
37)
   Primer pair: SEQ ID NOS: 61 and 90
   Size of PCR product: 382 by
38)
   Primer pair: SEQ ID NOS: 50 and 75
   Size of PCR product: 477 by
39)
   Primer pair: SEQ ID NOS: 60 and 80
   Size of PCR product: 558 by
40)
   Primer pair: SEQ ID NOS: 64 and 72
   Size of PCR product: 495 by
41)
   Primer pair: SEQ ID NOS: 45 and 76
   Size of PCR product: 552 by
42)
   Primer pair: SEQ ID NOS: 63 and 76
   Size of PCR product: 492 by
43)
   Primer pair: SEQ ID NOS: 60 and 91
   Size of PCR product: 400 by
44)
   Primer pair: SEQ ID NOS: 44 and 82
   Size of PCR product: 672 by
45)
   Primer pair: SEQ ID NOS: 41 and 92
   Size of PCR product: 435 by
46)
   Primer pair: SEQ ID NOS: 65 and 93
   Size of PCR product: 392 by
47)
   Primer pair: SEQ ID NOS: 67 and 94
   Size of PCR product: 460 by
48)
   Primer pair: SEQ ID NOS: 68 and 95
   Size of PCR product: 358 by

In the detection set according to the present invention, a single or multiplex real-time PCR may be performed using at least two primers specific to the kind of the HLA alleles and a fluorescent probe as one set, or using at least one combination thereof.

Also, the present invention relates to a selection kit for typing HLA alleles including a detection kit according to the present invention.

The HLA alleles which may be typed using the selection kit for typing HLA alleles according to the present invention may be HLA-A or HLA-B alleles.

The primer pair and the fluorescent probe are packaged into one reaction container, strip or microplate, and packaged using methods known in the art. Also, the selection kit according to the present invention may further include at least one selected from the group consisting of Taq polymerase, a reaction buffer containing 20 MgCl₂, dNTP and a stabilizer, and also further include another reagent known in the art, for example, a mater mix for real-time PCR.

When the selection kit for typing HLA alleles according to the present invention is used, a major histocompatibility complex may be detected at a genetic level. Therefore, the selection kit according to the present invention may be widely used to determine the histocompatibility more accurately.

Also, the present invention relates to a method for typing HLA alleles. Here, the method includes performing a real-time PCR on DNA isolated from a sample using a detection set for detecting HLA alleles according to the present invention, and typing the HLA alleles by checking the real-time PCR results.

Respective processes of the method for typing HLA alleles according to the present invention will be described in further detail, as follows.

Process 1 is performed to construct primers for amplification of HLA alleles. A primer is constructed to have a length of 17 to 24 nt using a locus showing the diversity of the HLA alleles as the starting point.

Preferably, at least two primers selected from sequences set forth in SEQ ID NOS: 1 to 34 may be used to selectively amplify the HLA-A alleles.

The primers may further include a primer pair having sequences set forth in SEQ ID NOS: 35 and 36 for amplifying an internal positive-control gene to enhance test reliability and check the status of a template in all PCR tubes and the PCR conditions used in this experimental procedure.

Process 2 is performed to amplify the primers constructed in Process 1 for constructing a fluorescent probe, and construct one or two fluorescent probes, which are able to detect the PCR product, for every HLA gene. The fluorescent probes are constructed to have a length of 28 to 35 nt by selecting a polymorphism-free locus on the HLA gene.

Particularly, the fluorescent probe may have a sequence set forth in SEQ ID NO: 37 or 38, which is able to detect amplification of the HLA-A alleles. Also, the fluorescent probe may have a sequence set forth in SEQ ID NO: 96 or 97, which is able to detect amplification of the HLA-B alleles.

In addition, the fluorescent probe may further include a fluorescent probe having a sequence set forth in SEQ ID NO: 98 for detecting amplification of a positive-control gene.

Process 3 is performed to amplify the primers using the real-time PCR and determine amplification of the HLA alleles in real time using a fluorescent probe at the same time.

In the method for typing HLA alleles according to the present invention, the single or multiplex real-time PCR may be performed under conventional reaction conditions. Also, the single real-time PCR and the multiplex real-time PCR may be performed under the same reaction conditions. In one example, initial denaturation is carried out at 95° C. for 1 minute, and a cycle including denaturation (at 95° C. for 25 seconds), annealing (at 65° C. for 45 seconds) and extension (at 72° C. for 30 seconds) may be performed a total of 40 times. During the real-time PCR, the fluorescence generated by the fluorescent probe in the annealing and extension processes is measured.

Also, the method for typing HLA alleles according to the present invention may be performed using a conventional real-time PCR method or device. The real-time PCR method is performed in real time in every PCR to detect and quantify the fluorescence, using DNA polymerase and a fluorescence resonance energy transfer (FRET) principle. Such a method may distinguish a specific PCR product from a non-specific PCR product and easily obtain the analysis results as an automated profile.

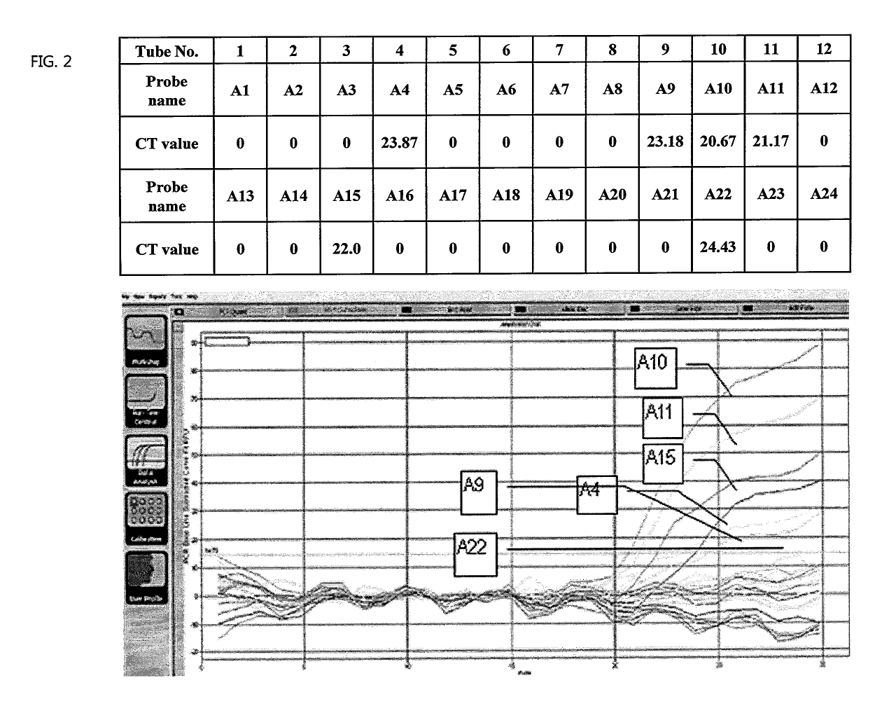

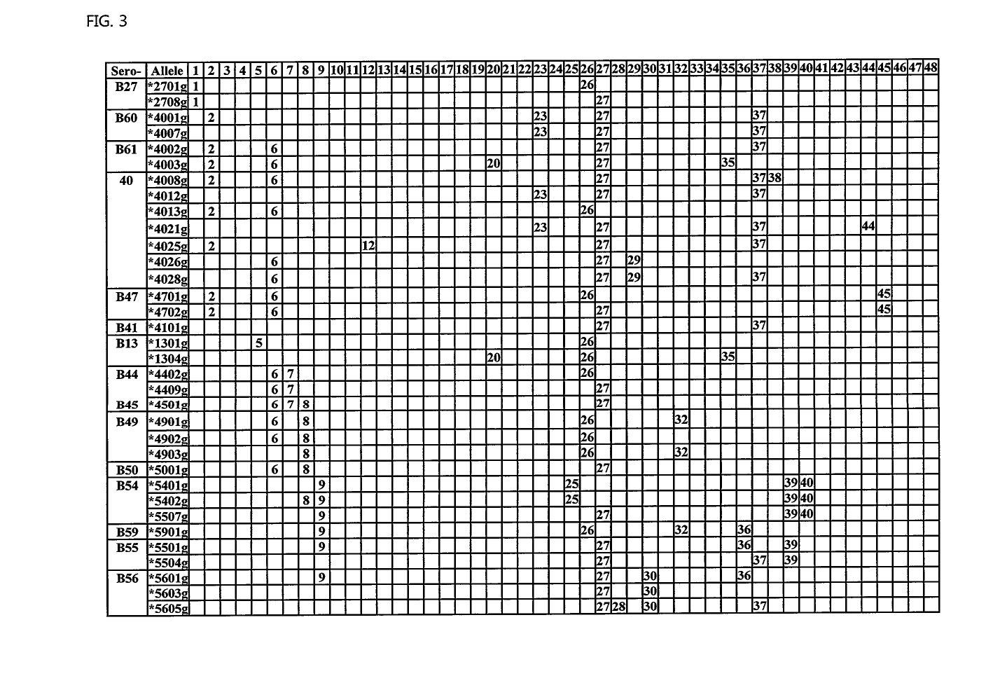

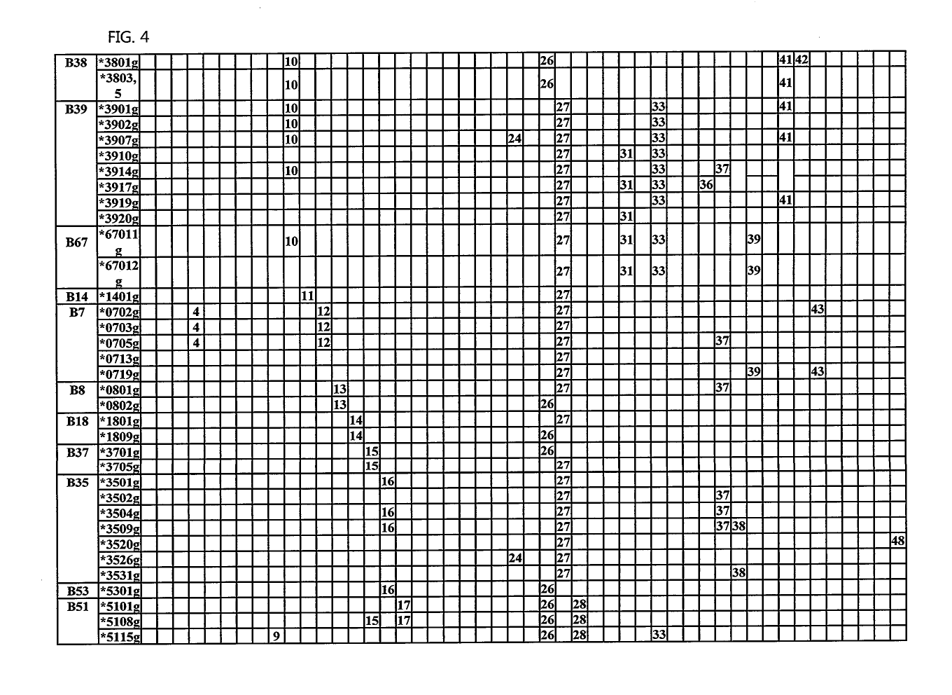

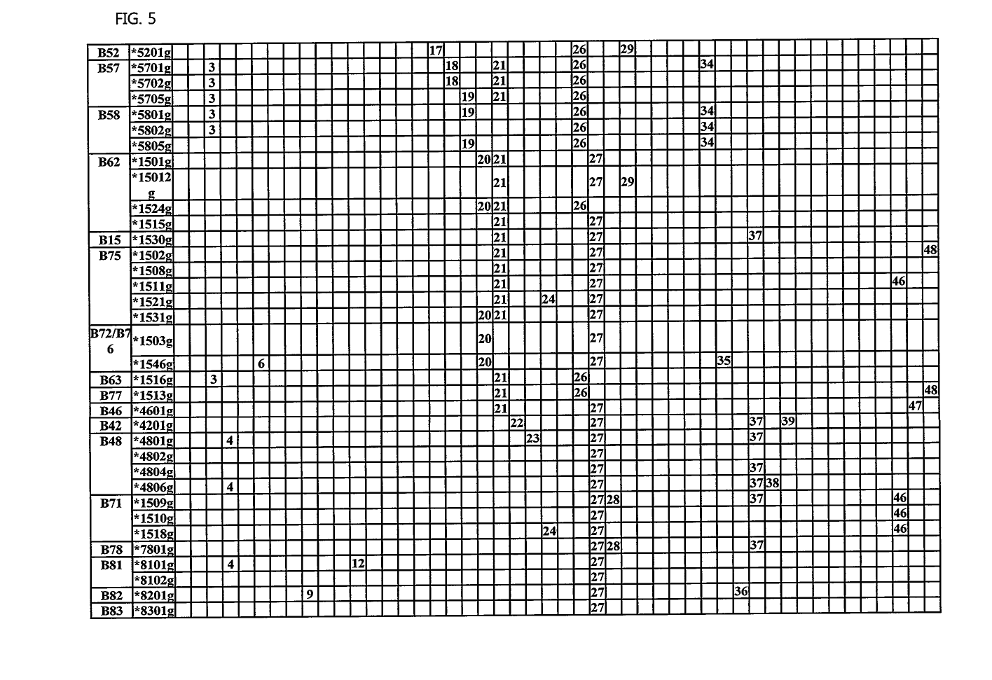

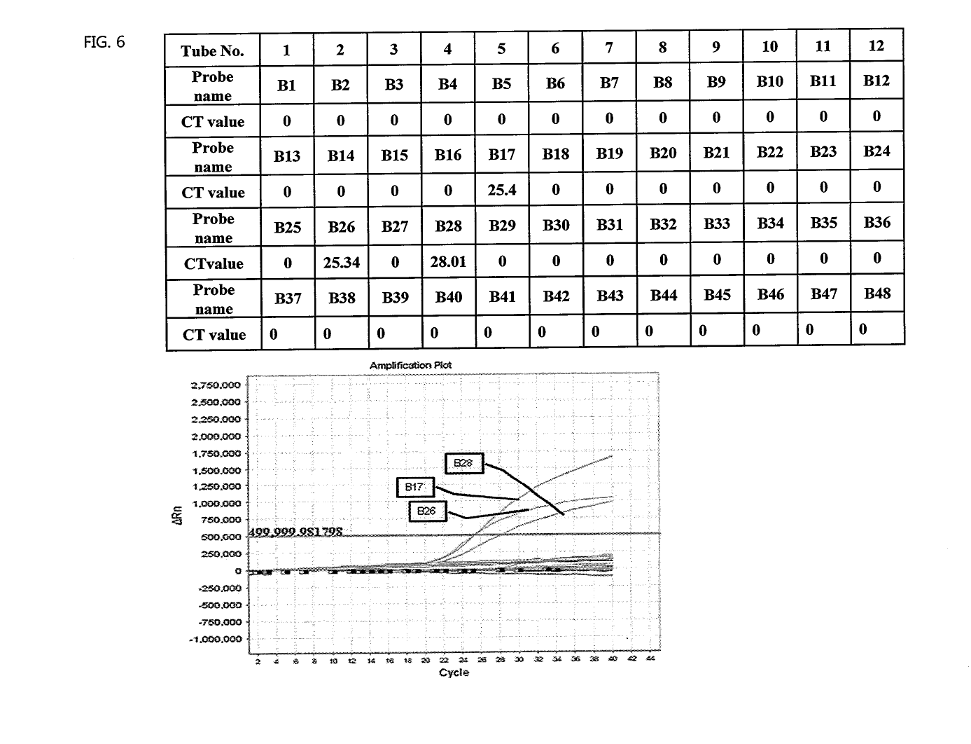

The real-time PCR device which may be used herein includes, but is not limited to, Real-time PCR device 7900, 7500 and 7300 series commercially available from AB, LightCycler®80 from Roche, Mx3000p from Stratagene, and a Chromo 4 device from BioRad, etc. When the PCR is terminated, a laser of such a real-time PCR device detects a fluorescent marker labeled in the fluorescent probe of the amplified PCR product to embody peaks as shown in FIGS. 2 and 6. Therefore, a program installed inside the PCR device is operated to automatically analyze the results without performing the electrophoresis.

In one example in the present invention, KoRAS™ (Kogene Biotech Co. Ltd.) may be used as an automated program. When the automated program is used, the analyzed results may be represented by an 0/X type or a Ct type. Therefore, it is possible to those unskilled in the art to easily appreciate the analyzed results.

The detection set for detecting HLA alleles according to the present invention and the method for typing HLA alleles using the same may significantly reduce conventional PCR procedures for detecting HLA alleles, and rapidly check the results in real time as well. Such a method may determine a small concentration of DNA fragments or a trace (less than 100 bp) of a PCR product, which could not be easily detected using an electrophoresis method, has no possibility to contaminate the PCR product, and all of its procedures are operated by an automated system when the results are analyzed and the test is terminated. Therefore, the method has low probability to cause experimenter's mistakes, errors in the test, etc., and is very useful in easily collecting and analyzing data and minimizing the used test time and manpower.

Hereinafter, the present invention will be described in detail with reference to Examples according to the present invention and Comparative Example not according to the present invention. However, the present invention is not limited to the following Examples.

### Example 1

### Construction of Primers for Amplification of Specific HLA-A Alleles

Primers having 17 mers to 24 mers were constructed using a locus showing the diversity of the HLA-A alleles as the 3′ terminus. Base sequences used for the respective primers are as follows:

Primer 1:
(SEQ ID NO: 1)
GGA GTA TTG GGA CCG GAA C
Primer 2:
(SEQ ID NO: 2)
GTG GCC CCT GGT ACC CGT
Primer 3:
(SEQ ID NO: 3)
ACG GAA TGT GAA GGC CCA G
Primer 4:
(SEQ ID NO: 4)
CCT CCA GGT AGG CTC TCA A
Primer 5:
(SEQ ID NO: 5)
AGC GAC GCC GCG AGC CA
Primer 6:
(SEQ ID NO: 6)
CGT CGT AGG CGT CCT GCC
Primer 7:
(SEQ ID NO: 7)
GGC CGG AGT ATT GGG ACG A
Primer 8:
(SEQ ID NO: 8)
CTG GTA CCG GCG GAG GAG
Primer 9:
(SEQ ID NO: 9)
GAT AGA GCA GGA GAG GCC T
Primer 10:
(SEQ ID NO: 10)
CGG AAT GTG AAG GCC CAC T
Primer 11:
(SEQ ID NO: 11)
CCC GGC CCG GCA GTG GA
Primer 12:
(SEQ ID NO: 12)
GTG GAT AGA GCA GGA GGG T
Primer 13:
(SEQ ID NO: 13)
ATG TAA TCC TTG CCG TCG TAA
Primer 14:
(SEQ ID NO: 14)
GGA CCA GGA GAC ACG GAA TA
Primer 15:
(SEQ ID NO: 15)
CAC TCC ACG CAC GTG CCA
Primer 16:
(SEQ ID NO: 16)
TCA CAG ACT GAC CGA GAG AG
Primer 17:
(SEQ ID NO: 17)
AGC GCA GGT CCT CGT TCA A
Primer 18:
(SEQ ID NO: 18)
CCG TCG TAG GCG TGC TGT
Primer 19:
(SEQ ID NO: 19)
CAC GCA OTT CGT GCG GTT T
Primer 20:
(SEQ ID NO: 20)
CTC TCT GCT GCT CCG CCG
Primer 21:
(SEQ ID NO: 21)
CAA GAG CGC AGG TCC TCG
Primer 22:
(SEQ ID NO: 22)
CAA GAG CGC AGG TCC TCG
Primer 23:
(SEQ ID NO: 23)
CGG AAT GTG AAG GCC CAG T
Primer 24:
(SEQ ID NO: 24)
CCT CCA GGT AGG CTC TCT G
Primer 25:
(SEQ ID NO: 25)
CCG AGT GGA CCT GGG GAC
Primer 26:
(SEQ ID NO: 26)
GAG CCA CTC CAC GCA CGT
Primer 27:
(SEQ ID NO: 27)
ACT CAC AGA CTG ACC GAG C
Primer 28:
(SEQ ID NO: 28)
AGG ATG GAG CCG CGG GCA
Primer 29:
(SEQ ID NO: 29)
AGG TAT CTG CGG AGC CCG
Primer 30:
(SEQ ID NO: 30)
TCC TCG TCC CCA GGC TCT
Primer 31:
(SEQ ID NO: 31)
GAG CCA CTC CAC GCA CCG
Primer 32:
(SEQ ID NO: 32)
ACC TGC GGA TCG CGC TCC G
Primer 33:
(SEQ ID NO: 33)
GGG TAC CAG CAG GAC GCT
Primer 34:
(SEQ ID NO: 34)
GGA CCA CTC CAC GCA CTC

Also, primers 35 and 36 were constructed to determine amplification of a positive-control reference gene (Homo sapiens adenomatous polyposis coli (APC)).

Primer 35:
(SEQ ID NO: 35)
ATG ATG TTG ACC TTT CCA GGG
Primer 36:
(SEQ ID NO: 36)
ATT CTG TAA CTT TTC ATC AGT TGC

### Example 2

### Construction of Primers for Amplification of Specific HLAB Alleles

Primers having 17 mers to 24 mers were constructed using a locus showing the diversity of the HLA-B alleles as the 3′ terminus. Base sequences used for the respective primers are as follows:

Primer 39:
(SEQ ID NO: 39)
GGC GCC GTG GAT AGA GCA A
Primer 40:
(SEQ ID NO: 40)
CCA CTC CAT GAG GTA TTT CC
Primer 41:
(SEQ ID NO: 41)
CGC CAC GAG TCC GAG GAA
Primer 42:
(SEQ ID NO: 42)
CGC GAG TCC GAG AGA GGA
Primer 43:
(SEQ ID NO: 43)
GCC GCG AGT CCG AGG AC
Primer 44:
(SEQ ID NO: 44)
CGC GAG TCC GAG GAT GGC
Primer 45:
(SEQ ID NO: 45)
GAC CGG AAC ACA CAG ATC TG
Primer 46:
(SEQ ID NO: 46)
ACC GGG AGA CAC AGA TCT G
Primer 47:
(SEQ ID NO: 47)
ACC GGG AGA CAC AGA TCT C
Primer 48:
(SEQ ID NO: 48)
GGA GTA TTG GGA CCG GAA C
Primer 49:
(SEQ ID NO: 49)
GAA CAT GAA GGC CTC CGC G
Primer 50:
(SEQ ID NO: 50)
GAC CGG AAC ACA CAG ATC TT
Primer 51:
(SEQ ID NO: 51)
GAC GAC ACC CAG TTC GTG A
Primer 52:
(SEQ ID NO: 52)
TAC CGA GAG AAC CTG CGC
Primer 53:
(SEQ ID NO: 53)
GAG CAG GAG GGG CCG GAA
Primer 54:
(SEQ ID NO: 54)
GGC CGG AGT ATT GGG ACG
Primer 55:
(SEQ ID NO: 55)
GAC GAC ACG CAG TTC GTG A
Primer 56:
(SEQ ID NO: 56)
GAC GAC ACG CTG TTC GTG A
Primer 57:
(SEQ ID NO: 57)
GAC GGC ACC CAG TTC GTG A
Primer 58:
(SEQ ID NO: 58)
GCG GGC GCC GTG GGT G
Primer 59:
(SEQ ID NO: 59)
GAC CGG AAC ACA CAG ATC TA
Primer 60:
(SEQ ID NO: 60)
CAG ATC TAC AAG GCC CAG G
Primer 61:
(SEQ ID NO: 61)
CCG AGA GAG CCT GCG GAA
Primer 62:
(SEQ ID NO: 62)
ACC GAG AGA ACC TGC GGA T
Primer 63:
(SEQ ID NO: 63)
GCG CTC CGC TAC TAC AAC
Primer 64:
(SEQ ID NO: 64)
ACG CCG CGA GTC CGA CAG G
Primer 65:
(SEQ ID NO: 65)
CTC CTG CTG CTC TCG GGA
Primer 66:
(SEQ ID NO: 66)
CGC CGC GAG TCC GAG AGA
Primer 67:
(SEQ ID NO: 67)
GAG ACA CAG AAG TAC AAG CG
Primer 68:
(SEQ ID NO: 68)
ACC GGA ACA CAC AGA TCT C
Primer 69:
(SEQ ID NO: 69)
CCT CCA GGT AGG CTC TGT C
Primer 70:
(SEQ ID NO: 70)
GGA GGA GGC GCC CGT CG
Primer 71:
(SEQ ID NO: 71)
CCT TGC CGT CGT AGG CGG
Primer 72:
(SEQ ID NO: 72)
ATC CTT GCC GTC GTA GGC T
Primer 73:
(SEQ ID NO: 73)
CGT TCA GGG CGA TGT AAT CT
Primer 74:
(SEQ ID NO: 74)
GCC GCG GTC CAG GAG CT
Primer 75:
(SEQ ID NO: 75)
GAG CCG CCG TGT CCG CG
Primer 76:
(SEQ ID NO: 76)
CGT GCC CTC CAG GTA GGT
Primer 77:
(SEQ ID NO: 77)
GAG CCA CTC CAC GCA CTC
Primer 78:
(SEQ ID NO: 78)
GAG CCA CTC CAC GCA CAG
Primer 79:
(SEQ ID NO: 79)
CCA GGT ATC TGC GGA GCG
Primer 80:
(SEQ ID NO: 80)
GAG CCA CTC CAC GCA CGT
Primer 81:
(SEQ ID NO: 81)
CCG CGC GCT CCA GCG TG
Primer 82:
(SEQ ID NO: 82)
TAC CAG CGC GCT CCA GCT
Primer 83:
(SEQ ID NO: 83)
GGG CCG CCT CCC ACT TGA
Primer 84:
(SEQ ID NO: 84)
CGT CGC AGC CAT ACA TCC A
Primer 85:
(SEQ ID NO: 85)
GCC ATA CAT CCT CTG GAT GA
Primer 86:
(SEQ ID NO: 86)
CGT CGC AGC CAT ACA TCA C
Primer 87:
(SEQ ID NO: 87)
CGC TCT GGT TGT AGT AGC C
Primer 88:
(SEQ ID NO: 88)
CGC TCT GGT TGT AGT AGC G
Primer 89:
(SEQ ID NO: 89)
CGC GCG CTG CAG CGT CTC
Primer 90:
(SEQ ID NO: 90)
GTC GTA GGC GTA CTG GTT
Primer 91:
(SEQ ID NO: 91)
GTC GTA GGC GTA CTG GTC
Primer 92:
(SEQ ID NO: 92)
CAA ACA TCC TCT GGA GGG T
Primer 93:
(SEQ ID NO: 93)
AGT CTG TGT GTT GGT CTT GT
Primer 94:
(SEQ ID NO: 94)
GCC GCG GTC CAG GAG CT
Primer 95:
(SEQ ID NO: 95)
GCC ATA CAT CCT CTG GAT GA

### Example 3

### Construction of Fluorescent Probe for Determining Amplification

In order to determine amplification of an HLA-A gene, one universal fluorescent probe was constructed, and one universal fluorescent probe which was able to determine amplification of a positive-control reference gene was constructed. Base sequences and fluorescent materials used for the respective fluorescent probes are constructed as follows:

HLA-A fluorescent probe 1:
(SEQ ID NO: 37)
FAM 5′-CCC GGT TTC ATT TTC AGT TTA GGC CAA AAA
T 3′ BHQ1
HLA-A fluorescent probe 2:
(SEQ ID NO: 38)
FAM 5′-GTT CTC ACA CCV(G/A/C)TCC AGA K(G/T)R
(A/G)A TGTD(G/A/T)TG GCT 3′ BHQ1
HLA-B fluorescent probe 3:
(SEQ ID NO: 96)
FAM 5′-ACC CTC GAC CGG CGA GAG CCC CAG GCG
CG 3′ BHQ1
HLA-B fluorescent probe 4:
(SEQ ID NO: 97)
FAM 5′-AGA GCA R(A/G)GA GGG GCC GGA R(A/G)TA TTG
GGA C 3′ BHQ1
Positive-control reference gene fluorescent
probe 2:
(SEQ ID NO: 98)
Cyanine 3 5′-ACA GAA CTA ACC TCC AAC CAA CAA TCA
GC 3′ BHQ2.

### Example 4

### HLA-A and HLA-B Allele Typing Using Real-Time PCR

Total genomic DNA was isolated from a human blood sample, and the isolated DNA, 2 mM dATP, dGTP, dCTP and d′FTP, a specific primer pair which is able to amplify HLA-A and HLA-B alleles, a primer pair for amplifying a positive-control reference gene, HLA-A and -B fluorescent probes for determining amplification of the. HLA-A and HLA-B alleles, a positive-control reference gene fluorescent probe, a polymerization reaction buffer, and polymerase were added to a PCR tube, and PCR was performed on the resulting PCR mix once at 96° C. for 1 minute, then performed 40 times at 96° C. for 25 seconds, at 65° C. for 45 seconds, and at 72° C. for 30 seconds, using a real-time PCR device. Thereafter, the real-time amplification was determined during the extension at 72° C. for 30 seconds. The amplification in the real-time PCR device was determined at 490 nm and 532 nm using FAM and Cyanine, respectively. A concentration of each of the specific primers used herein was 1.0 gM, and a concentration of the positive control was 0.5 11M. Also, the used PCR buffer includes 67 mM Tris base, 16.6 mM ammonium sulphate, 0.1% Tween, and 0.2 mM MgCl₂. A concentration of the used dNTP was 0.2 mM, and Taq polymerase (Biotools) was used. The used real time PCR device was a device commercially available from Bio-Rad.

As shown in FIG. 2, the number of tubes in which the starting point is observed was measured to be unkn-4 (23.87), unkn-9 (23.18), unkn-10 (20.67), unkn-11 (21.17), unkn-15 (22), and unkn-22 (24.43). Referring to the table showing these analyses, the unkn-4, unkn-15 and unkn-22 were then amplified, and confirmed to be HLA-A24(*2402g), and the unkn-9, unkn-10 and unkn-11 were amplified, and confirmed to be HLA-A33(*3301g).

As shown in FIG. 6, the number of tubes in which the starting point is observed was measured to be D5 Target1 (25.4), E2 Target1 (25.34) and E4 Target1 (28.1). Referring to the table showing these analyses, the D5 Target1 (25.4), E2 Target1 (25.34) and E4 Target1 (28.1) were then amplified, and confirmed to be HLA-B51(*5101g).

The selection kit for typing HLA alleles using a real-time PCR according to the present invention may be useful in amplifying a gene using a common primer specific to the HLA alleles instead of a fluorescent primer, typing the HLA alleles through a real-time PCR, without using electrophoresis, using a fluorescent probe which is able to detect amplification of the gene, significantly reducing the analysis costs, and easily typing the genes containing several single-base mutations.

While the invention has been shown and described with reference to predetermined exemplary embodiments thereof, it will be understood by those skilled in the art that various changes in form and details may be made therein without departing from the scope of the invention as defined by the appended claims.

What is claimed is:

1. A detection set for detecting HLA alleles using a real-time polymerase chain reaction (PCR) comprising:
primer sets which are able to specifically amplify the HLA alleles, and
a fluorescent probe which is able to detect amplification of the HLA alleles;
wherein the primer sets are primer sets specific to HLA-A*01 set forth in SEQ ID NOS: 14 and 29; primer sets specific to HLA-A*02 set forth in SEQ ID NOS: 30 and 2; primer sets specific to HLA-A*23 or HLA-A*24 set forth in SEQ ID NOS: 7 and 4; primer sets specific to HLA-A*24 set forth in SEQ ID NOS: 32 and 24; primer sets specific to HLA-A*25, HLA-A*26, HLA-A*34 or HLA-A*66 set forth in SEQ ID NOS: 1 and 13; primer sets specific to HLA-A*68 or HLA-A*69 set forth in SEQ ID NOS 3 and 22; primer sets specific to HLA-A*29 set forth in SEQ ID NOS 28 and 17; primer sets specific to HLA-A*31 set forth in SEQ ID NOS: 9 and 17; primer sets specific to HLA-A*33 set forth in SEQ ID NOS: 1 and 17; primer sets specific to HLA-A*29, HLA-A*31, HLA-A*33 or HLA-A*74 set forth in SEQ ID NOS: 25 and 17; primer sets specific to HLA-A*01, HLA-A*03, HLA-A*25, HLA-A*26, HLA-A*34, HLA-A*66, HLA-A*43, HLA-A*11, HLA-A*29, HLA-A*30, HLA-A*31, HLA-A*32, HLA-A*33, HLA-A*74 or HLA-A*80 set forth in SEQ ID NOS: 10, 23 and 21; primer sets specific to HLA-A*25, HLA-A*26 or HLA-A*43 set forth in SEQ ID NOS: 33 and 31; primer sets specific to HLA-A*36 set forth in SEQ ID NOS: 14 and 26; primer sets specific to HLA-A*03 or HLA-A*24 set forth in SEQ ID NOS: 5 and 15; primer sets specific to HLA-A*24 set forth in SEQ ID NOS: 7 and 24; primer sets specific to HLA-A*26 or HLA-A*43 set forth in SEQ ID NOS: 27 and 13; primer sets specific to HLA-A*11 set forth in SEQ ID NOS: 3 and 20; primer sets specific to HLA-A*68 set forth in SEQ ID NOS: 1 and 6; primer sets specific to HLA-A*30 set forth in SEQ ID NOS: 11 and 18; primer sets specific to HLA-A*32 set forth in SEQ ID NOS: 16 and 17; primer sets specific to HLA-A*32 or HLA-A*74 set forth in SEQ ID NOS: 19 and 8; primer sets specific to HLA-A*02, HLA-A*24, HLA-A*26, HLA-A*68 or HLA-A*69 set forth in SEQ ID NOS: 10, 23 and 22; primer sets specific to HLA-A*02 set forth in SEQ ID NOS: 12 and 4; primer sets specific to HLA-A*66 set forth in SEQ ID NOS: 33 and 34; primer sets specific to B*27(*2701g) set forth in SEQ ID NOS: 46 and 77; primer sets specific to B60(*4001g), B61(*4002g), B40(*4008g) or B47(*4701g) set forth in SEQ ID NOS: 41 and 75; primer sets specific to B57(*5701g), B58(*5801g) or B63(*1516g) set forth in SEQ ID NOS: 49 and 78; primer sets specific to B7(*0702g), B48(*4801g) or B81(*8101g) set forth in SEQ ID NOS: 42 and 82; primer sets specific to B13(*1301g)) set forth in SEQ ID NOS: 52 and 83; primer sets specific to B61(*4002g), B40(*4008g), B47(*4701g), B44(*4402g), B45(*4501g), B49(*4901g) or B50(*5001g) set forth in SEQ ID NOS: 41 and 89; primer sets specific to B44(*4402g) or B45(*4501g) set forth in SEQ ID NOS: 47 and 79; primer sets specific to B45(*4501g), B49(*4901g) or B50(*5001g) set forth in SEQ ID NOS: 40 and 72; primer sets specific to B54(*5401g), B59(*5901g), B55(*5001g), B56(5601g) or B82(*8201g) set forth in SEQ ID NOS: 48 and 72; primer sets specific to B38(*3801g), B39(*3901g) or B67(*6701g) set forth in SEQ ID NOS: 53 and 76; primer sets specific to B14(*1401g) set forth in SEQ ID NOS: 53 and 84; primer sets specific to B40(*4025g), B7(*0702g) or B81(*8101g) set forth in SEQ ID NOS: 48 and 82; primer sets specific to B8(*0801g) set forth in SEQ ID NOS: 50 and 81; primer sets specific to B18(*1801g) set forth in SEQ ID NOS: 39 and 74; primer sets specific to B37(*3701g) or B51(*5108g) set forth in SEQ ID NOS: 43 and 69; primer sets specific to B35(*3501g) or B53(*5301g) set forth in SEQ ID NOS: 50 and 70; primer sets specific to B51(*5101g) or B52(*5201g) set forth in SEQ ID NOS: 43 and 73; primer sets specific to B57(*5701g) set forth in SEQ ID NOS: 54 and 86; primer sets specific to B57(*5705g) or B58(*5801g) set forth in SEQ ID NOS: 49 and 85; primer sets specific to B13(*1304g), B61(*4003g), B62(*1501g), B72(*1503g) or B76(*1512g) set forth in SEQ ID NOS: 47 and 71; primer sets specific to B57(*5701g), B62(*1501g), B75(*1502g), B63(*1516g), B77(*1513g) or B46(*4601g) set forth in SEQ ID NOS: 44 and 78; primer sets specific to B42(*4201g) set forth in SEQ ID NOS: 51 and 81; primer sets specific to B60(*4001g) or B48(*4801g) set forth in SEQ ID NOS: 47 and 82; primer sets specific to B39(*3907g) or B75(*1521g) set forth in SEQ ID NOS: 45 and 71; primer sets specific to B54(*5401g) set forth in SEQ ID NOS: 58 and 74; primer sets specific to B27(*2701g), B47(*4701g), B13(*1301g), B44(*4402g), B49(*4901g), B59(*5901 g), B38(*3801g), B8(*0802g), B18(*1809g), B37(*3701g), B53(*5301g), B51(*5101g), B52(*5201g), B57(*5701g), B58(*5801g), B63(*1516g), B77(*1513g) or B62(*1524g) set forth in SEQ ID NOS: 55, 56, 51, 57 and 88; primer sets specific to B27(*2708g), B60(*4001g), B61(*4002g), B40(*4008g), B47(*4702g), B41(*4101g), B44(*4409g), B45(*4501g), B50(*5001g), B55(*5501g), B56(*5601g), B39(*3901g), B67(*6701g), B14(*1401g), B7(*0702g), B8(*0801g), B18(*1801g), B37(*3705g), B35(*3501g), B62(*1501g), B15(*1530g), B72(*1503g), B76(*1512g), B46(*4601g), B42(*4201g), B48(*4801g), B71(*1509g), B78(*7801g), B81(*8101g), B82(*8201g) or B83(*8301g) set forth in SEQ ID NOS: 55, 56, 51, 57 and 87; primer sets specific to B56(*5605g), B51(*5101g), B71(*1509g) or B78(*7801g) set forth in SEQ ID NOS: 48 and 73; primer sets specific to B40(*4026g), B52(*5201g) or B62(*15012) set forth in SEQ ID NOS: 47 and 73; primer sets specific to B56(*5601g) set forth in SEQ ID NOS: 60 and 78; primer sets specific to B39(*3910g) or B67(*67011g) set forth in SEQ ID NOS: 59 and 76; primer sets specific to B49(*4901g) or B59(*5901g) set forth in SEQ ID NOS: 62 and 72; primer sets specific to B39(*3901g), B67(*6701g) or B51(*5115g) set forth in SEQ ID NOS: 61 and 76; primer sets specific to B57(*5701g) or B58(*5801g) set forth in SEQ ID NOS: 54 and 71; primer sets specific to B61(*4003g), B13(*1304g) or B72(*1546g) set forth in SEQ ID NOS: 41 and 71; primer sets specific to B59(*5901g), B55(*5501g), B56(*5601g), B39(*3917g) or B82(*8201g) set forth in SEQ ID NOS: 66 and 72; primer sets specific to B60(*4001g), B62(*4002g), B40(*4008g), B41(*4101g), B55(*5504g), B56(*5605g), B8(*0801g), B35(*3502g), B15(*1530g), B42(*4201g), B48(*4801g), B71(*1509g) or B78(*7801g) set forth in SEQ ID NOS: 61 and 90; primer sets specific to B40(*4008g), B35(*3509g) or B48(*4806g) set forth in SEQ ID NOS: 50 and 75; primer sets specific to B54(*5401g), B55(*5501g), B67(*6701g), B7(*0719g) or B42(*4201g) set forth in SEQ ID NOS: 60 and 80; primer sets specific to B54(*5401g) set forth in SEQ ID NOS: 64 and 72; primer sets specific to B38(*3801g) or B39(*3901g) set forth in SEQ ID NOS: 45 and 76; primer sets specific to B38(*3801g) set forth in SEQ ID NOS: 63 and 76; primer sets specific to B7(*0702g) set forth in SEQ ID NOS: 60 and 91; primer sets specific to B40(*4021g) set forth in SEQ ID NOS: 44 and 82; primer sets specific to B47(*4701g) set forth in SEQ ID NOS: 41 and 92; primer sets specific to B71(*1509g) or B75(*1511g) set forth in SEQ ID NOS: 65 and 93; primer sets specific to B46(*4601g) set forth in SEQ ID NOS: 67 and 94; and primer sets specific to B35(*3520g), B75(*1502g) or B77(*1513g) set forth in SEQ ID NOS: 68 and 95; and
wherein the fluorescent probe is at least one selected from the group consisting of sequences set forth in SEQ ID NOS: 37, 38, 96 and 97, which amplification of the HLA-A alleles is detected by one or two of two fluorescent probes set forth in SEQ ID NOS: 37 and 38, and amplification of the HLA-B alleles is detected by one or two of two fluorescent probes set forth in SEQ ID NOS: 96 and 97.

4. The detection set according to claim 1, wherein the fluorescent probe has a 5′ terminus labeled with one fluorescent marker and a 3′ terminus labeled with one fluorescence quencher and a molecular grove binding non-fluorescence quencher.

5. The detection set according to claim 1, further comprising a primer having sequences set forth in either SEQ ID NOS: 35 and/or 36 to amplify a positive control gene.

6. The detection set according to claim 1, further comprising a fluorescent probe having one or more sequences set forth in SEQ ID NO: 98 to detect amplification of the positive control gene.

7. The detection set according to claim 1, wherein the real-time PCR is a single or multiplex PCR using at least one primer pair and the fluorescent probe.

8. A selection kit for typing HLA alleles, comprising a detection set defined in claim 1.

9. The selection kit according to claim 8, wherein the HLA alleles are HLAA or HLA-B alleles.

10. A method for typing HLA alleles, comprising: performing a real-time PCR on DNA isolated from a sample using a detection set for detecting HLA alleles defined in claim 1; and typing the HLA alleles by checking the real-time PCR results.

11. The method according to claim 10, wherein the real-time PCR is a single or multiplex PCR.
